(19) United States
(12) Patent Application Publication
(10) Pub. No.: US 2006/0270711 A1
(43) Pub. Date: Nov. 30, 2006

(54) Tenatoprazole salts and process of preparation thereof

(75) Inventors: Ramesh Anna Joshi, Maharashtra (IN); Rohini Ramesh Joshi, Maharashtra (IN); Vijay Naryan Wakchaure, Maharashtra (IN); Mukund Keshav Gurjar, Maharashtra (IN)

Correspondence Address: NIXON & VANDERHYE, PC, 901 NORTH GLEBE ROAD, 11TH FLOOR, ARLINGTON, VA, 22203, US

(73) Assignee: Council of Scientific and Industrial Research, New Delhi (IN)

(21) Appl. No.: 11/490,247

(22) Filed: Jul. 21, 2006

Related U.S. Application Data
(62) Division of application No. 11/175,027, filed on Jul. 6, 2005.
Application No. 11/175,027 is a continuation of application No. 10/973,983, filed on Oct. 27, 2004, now abandoned.

(51) Int. Cl. A61K 31/4439; C07D 403/02

(52) U.S. Cl. 514/338; 546/273.7

(57) ABSTRACT

The present invention relates to of salts of 5-methoxy-2-(4-methoxy-3,5-dimethylpyridin-2-ylmethylsulfinyl) imidazo[4,5-b]pyridine (Tenatoprazole) of formula (1) 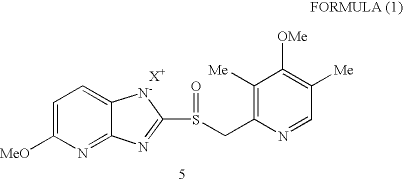
wherein X is Li, Na, Ca, K or Mg and to a process for the preparation thereof which comprises oxidizing a compound of formula (2) and isolating the salt (Li, Na) by treatment with the alkali hydroxide or exchanging the sodium salt of tenatoprazole with Mg⁺⁺ or Ca⁺⁺ cation.

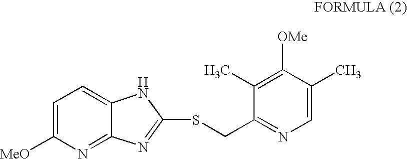

### FIELD OF THE INVENTION

The present invention relates to of salts of 5-methoxy-2-(4-methoxy-3,5-dimethylpyridin-2-ylmethylsulfinyl) imidazo[4,5-b]pyridine (Tenatoprazole) and to a process for the preparation thereof. The Tenatoprazole salts of the invention have the formula (1) 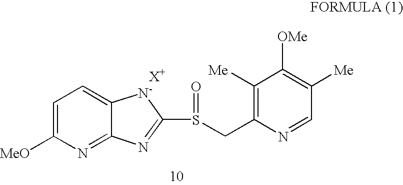
wherein X is Li, Na, Ca, K or Mg. More particularly the present invention relates to a process of preparing the salts of formula (1) by oxidizing a compound of formula (2).

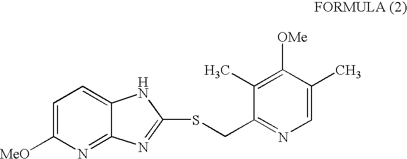

### BACKGROUND OF THE INVENTION

In the prior art U.S. Pat. No. 4,738,974 teaches the preparation of salts of omeprazole, mentions compositions consisting of salts of tenatoprazole however no details of the method of preparations of the said salts of tenatoprazole, their physical constants, characterization has been reported. Claim No. 14 of the PCT patent No. (PCT/SE94/00006 WO 95/18612) mentions along with others that the active substance TU 199 (Tenatoprazole) can be administered in the form of basic salts such as magnesium or sodium. Preparation of Tenatoprazole in free form as disclosed in patent EP-0254588 involves use of very dilute solution of sulfide in chloroform (100 ml per gram of sulfied) making it difficult for scale up.

### OBJECTS OF THE INVENTION

The main object of the present invention is to provide a process for the preparation of salts of Tenatoprozole having formula (1) wherein X is Li, Na, Ca, K or Mg.

Another object is to isolate salts of Tenatoprozole such as Na, K, Li from oxidation reaction mixture in single step thus avoiding the separation of free sulfinyl compund.

Yet another object is to prepare salts such as Ca, Mg from sodium salt of Tenatoprazole.

### SUMMARY OF THE INVENTION

Accordingly the present invention provides salts of 5-methoxy-2-(4-methoxy-3,5-dimethylpyridin-2-ylmethylsulfinyl) imidazo[4,5-b]pyridine (Tenatoprazole) of formula (1) 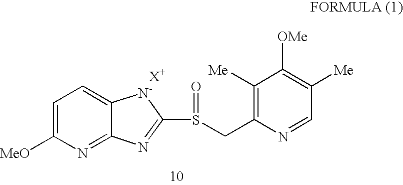
wherein X=Na, K, Li, Mg, Ca.

The present invention also provides a process for the preparation of 5-methoxy-2-(4-methoxy-3,5-dimethylpyridin-2-ylmethylsulfinyl) imidazo[4,5-b]pyridine (Tenatoprazole) salts of formula (1) 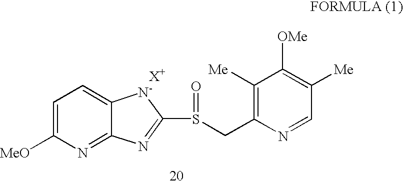
wherein X=Na, K, Li, Mg, Ca which comprises:
(a) oxidizing a sulfide compound of formula (2) 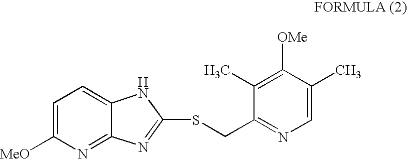
   in the presence of an organic solvent to obtain an organic layer;
(b) separating and washing the organic layer;
(c) extracting the organic layer with an aqueous alkali;
(d) washing the alkali extract with the solvent used in step (a);
(e) separating the aqueous layer and agitating at room temperature to obtain the compound of formula (1).

In one embodiment of the invention, in step (b), the organic layer is washed with saturated bicarbonate solution of sodium or potassium.

In one of embodiment of the invention the oxidizing agent is a peracid selected from the group consisting of m-chloroperbenzoic acid, perbenzoic acid and peracetic acid.

In another embodiment of the invention, the concentration of oxidizing agent is in the range of 1 to 1.3 mole per mole of sulfide of formula (2).

In another embodiment of the invention, the solvent used is a halogenated hydrocarbon selected from the group consisting of chloroform and terachloroethane; or an alcohol selected from the group consisting of methanol, ethanol, propanol and butanol; or a mixture of any two or more thereof.

In another embodiment of the invention the aqueous alkali is selected from the group consisting of hydroxides of Na, K and Li.

In yet another embodiment of the invention the Ca and Mg salts of compound of formula (1) are prepared by treating Na, K or Li salt of tenatoprazole with halides of Mg and Ca or Mg(OR)₂ Ca(OR)₂ wherein R is an alkyl group containing 1-4 carbon unit.

### DETAILED DESCRIPTION OF THE INVENTION

The present invention provides salts of 5-methoxy-2-(4-methoxy-3,5-dimethylpyridin-2-ylmethylsulfinyl) imidazo[4,5-b]pyridine (Tenatoprazole) of formula (1) 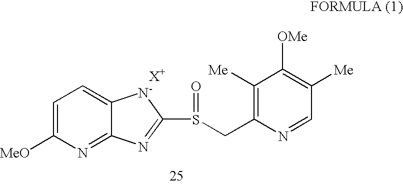
wherein X=Na, K, Li, Mg, Ca.
The salts of formula (1) are prepared by oxidizing a sulfide compound of formula (2) 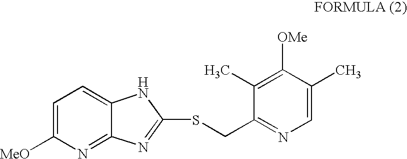
in the presence of an organic solvent. The organic layer so formed is then separated and washed, preferably with saturated bicarbonate solution of sodium or potassium, and then extracted with an aqueous alkali. The alkali extract is washed with the same solvent and the aqueous layer so obtained is separated and agitated preferably at room temperature to obtain the compound of formula (1).

The oxidizing agent is a peracid selected from the group consisting of m-chloroperbenzoic acid, perbenzoic acid and peracetic acid. The concentration of oxidizing agent is in the range of 1 to 1.3 mole per mole of sulfide of formula (2). The solvent used is a halogenated hydrocarbon selected from the group consisting of chloroform and terachloroethane; or an alcohol selected from the group consisting of methanol, ethanol, propanol and butanol; or a mixture of any two or more thereof. The aqueous alkali is selected from the group consisting of hydroxides of Na, K and Li. The Ca and Mg salts of compound of formula (1) are prepared by treating Na, K or Li salt of tenatoprazole with halides of Mg and Ca or Mg(OR)₂ Ca(OR)₂ wherein R is an alkyl group containing 1-4 carbon unit.

Tenatoprozole salts of Formula (I) are being extensively investigated clinically as a gastric acid secretion inhibiting agent.

A problem with this type of drugs i.e. proton pump inhibitors having methyl sulfinyl group, is their stability characteristics. Upon storage without any special precautions being taken they degrade at a rate, which is higher than desired. It is desirable to obtain physical forms which exhibit improved stability. This need for more stable form is apparent, considering the often longer time periods involved from the synthesis of the active substance through its incorporation in pharmaceutical preparations, distribution of the finished product to pharmacies etc. up to the consumption of the preparation by patient. The present invention provides such new salts/forms of tenatoprazole which exhibit improved storage stability.

The salts of the formula (1) are also easier to handle than the neutral form in the manufacture of pharmaceutical dosage unit.

Preferred salts are those wherein X is Na⁺, Mg²⁺. The Na⁺ salt is especially preferred for the preparation of liquid pharmaceutical formulations e.g. solutions for intravenous administration. The Mg²⁺ salts are especially preferred for the preparation of tablets.

In order to overcome it's low storage stability, different salts of tenatoprazole were prepared without isolating free base.

The salts of the invention are prepared by reacting tenatoprazole with a base capable of releasing the cation X wherein X⁽ⁿ⁺⁾ is as defined above to give a salt of the formula which salt is thereafter isolated.

The process of the present invention is described hereinbelow with reference to the examples which are illustrative only and should not be construed to limit the scope of the present invention in any manner.

### EXAMPLE 1

This example describes the preparation of 5-methoxy 2-[[(4-methoxy-3,5-dimethyl)-2-pyridyl methyl] sulfinyl] 1H imidazo [4,5-b] pyridine, Sodium Salt dihydrate:

To a solution of 5-methoxy 2-[[(4-methoxy-3,5-dimethyl)-2-pyridyl methyl] thio]1H imidazo [4,5-b]pyridine (20 gm.; 60.6 mmol.) in chloroform (400 ml), an aqueous solution of potassium bicarbonate (8 gm.; 79.9 mmol., in 60 ml H₂O) was added and cooled to 0-5° C. m-CPBA solution (20 gm; 55-75% in 130 ml Chloroform) was added drop wise over a period of 1 hr 25 min. at 0-5° C. with stirring. After complete addition reaction mixture was stirred at 0-5° C. for 20 min. The completion of the oxidation was followed by TLC (2% methanol in chloroform). Organic layer was separated and washed with saturated solution of potassium bicarbonate. Organic layer was extracted with sodium hydroxide solution (4.8 gm, NaOH in 130 ml H₂O) Alkaline extract again washed with chloroform (2×50 ml chloroform). Alkaline extract was depressurized on rotavapour to remove traces of chloroform. Aqueous layer then stirred for 20 minutes and precipitated solid was filtered off to get 21.5 gm of crude sodium salt. This was further purified by recrystallisation [diisopropyl ether (80 ml) and dimethyl formamide (53 ml)] to obtain pure sodium salt of tenatoprazole (16.8 gm, 76%) mp 224-226° C.

¹H NMR: (DMSO-d₆) 2.15(s, 3H), 2.20 (s,3H), 3.68 (s, 3H), 3.71 (s, 3H), 4.41 and 4.58 (AB-system, J=12.9 Hz, 2H), 6.56 (dd, J=8.5 Hz and 2.4 Hz, 1H), 7.00 (d, J=2.4 Hz, 1H), 7.34 (d, J=8.5 Hz), 8.30 (s, 1H).

Analysis-Calcd for C16H17N4O3S Na,2H2O: C, 47.5; H, 4.2; N, 13.86; S,7.92; Found: C, 46.61; H. 5.69; N,13.77; S,7.89.

### EXAMPLE 2

This example describes the preparation of 5-methoxy 2-[[(4-methoxy-3,5dimethyl)-2-pyridyl methyl] sulfinyl] 1H imidazo [4,5-b] pyridine, Potassium Salt:

To a solution of 5-methoxy 2-[[(4-methoxy-3,5-dimethyl)-2-pyridyl methyl] thio]1H imidazo [4,5-b]pyridine (1 gm, 3 mmol.) in chloroform (15 ml), an aqueous solution of potassium bicarbonate (0.2 gm.; 2 mmol, in 5 ml H₂O) was added and cooled to 0-5° C. m-CPBA solution (1 gm;. 55-75%. in 15 ml Chloroform) was added drop wise over a period of 1 hr 25 min. at 0-5° C. with stirring. After complete addition reaction mixture was stirred at 0-5° C. for 20 min. The completion of the oxidation was followed by TLC (2% methanol in chloroform). Organic layer was separated and washed with saturated solution of potassium bicarbonate. Organic layer was extracted with potassium hydroxide solution (0.160 gm. KOH in 6 ml H₂O) Alkaline extract again washed with chloroform (2×10 ml chloroform). Alkaline extract was depressurized on rotavapour to remove traces of chloroform. Aqueous layer then stirred for 20 minutes and filtered clear (celite). Evaporation to dryness gave a crystalline residue. Recrystallisation from Dimethyl formamide and Diisopropyl ether yielded tenatoprazole potassium salt (0.85 g, 69%), mp 159-161° C.

### EXAMPLE 3

This example describes the preparation of 5-methoxy 2-[[(4-methoxy-3,5-dimethyl)-2-pyridyl methyl] sulfinyl] 1H imidazo [4,5-b] pyridine, lithium Salt:

To a solution of 5-methoxy 2-[[(4-methoxy-3,5-dimethyl)-2-pyridyl methyl] thio]1H imidazo [4,5-b]pyridine (1 gm.; 3 mmol) in chloroform (40 ml), an aqueous solution of potassium bicarbonate (0.2 gm, 2 mmol, in 5 ml H₂O) was added and cooled to 0-5° C. m-CPBA solution (1 gm; 55-75%. in 15 ml Chloroform) was added drop wise over a period of 1 hr 25 min. at 0-5° C. with stirring. After complete addition reaction mixture was stirred at 0-5° C. for 20 min. The completion of the oxidation was followed by TLC (2% methanol in chloroform). Organic layer was separated and washed with saturated solution of potassium bicarbonate. Organic layer was extracted with lithium hydroxide solution (0.160 gm, LiOH in 10 ml H₂O) Alkaline extract again washed with chloroform (2×10 ml chloroform). Alkaline extract was depressurized on rotavapour to remove traces of chloroform. Aqueous layer then stirred for 20 minutes and precipitated solid was filtered off, dried. Recrystallisation from Dimethyl formamide and Diisopropyl ether and acetonitrile yielded tenatoprazole lithium salt (0.73 g, 71%) mp 196-197° C.

### EXAMPLE 4

This example describes preparation of di-tenatoprazole magnesium salt dihydrate:

Magnesium chloride (0.18 gm, 1.9 mmol) dissolved in deionised water (10 ml) was added drop wise under vigorous stirring to a solution of tenatoprzole sodium (1 gm, 2.7 mmol) in deionised water (30 ml) and then stirring was continued for 1 h. Precipitated solid was filtered off, dried, and then recrystallised in dimethyl fomamide and diisopropyl ether and acetonitrile yielded di-tenatoprazole magnesium salt (0.65 g, 61%) mp 190-191° C.

### EXAMPLE 5

This example describes preparation of di-tenatoprazole calcium salt dihydrate:

Calcium chloride (0.18 gm, 1.62 mmol) dissolved in deionised water (10 ml) was added drop wise under vigorous stirring to a solution of tenatoprazole sodium (1 gm, 2.7 mmol,) in deionised water (30 ml) and then stirring was continued for 1 h. at room temperature. Precipitated solid was filtered off washed with deionised water until no Cl⁻ was detectable (AgNO₃), dried, and recrystallised in dimethyl fomamide and diisopropyl ether and acetonitrile yielded di-tenatoprazole calcium salt (0.88 g, 80%) mp 194-196° C.

The main advantages of the process are:

1. The present invention provides the method for preparation of stable novel salts of formula (1) in a single step i.e. without isolating 5-methoxy-2-(4-methoxy-3,5-dimethylpyridin-2-ylmethylsulfinyl) imidazo [4,5-b] pyridine in free form from reaction mixture.

2. The invention also provides the method for preparation of Ca and Mg salts of Tenatoprazole from its sodium salt.

1. (canceled)

2. A process for the preparation of 5-methoxy-2-(4-methoxy-3,5-dimethylpyridin-2-ylmethylsulfinyl) imidazo[4,5-b]pyridine (Tenatoprazole salt of formula (1) 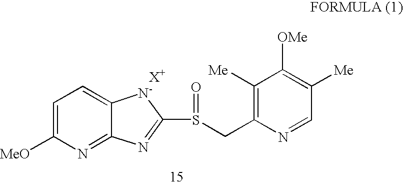
wherein X=Na, K, Li, Mg, Ca which comprises:
(a) oxidizing a sulfide compound of formula (2) 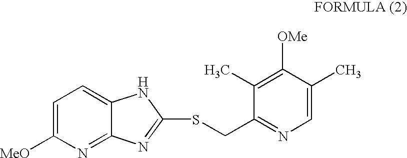
in the presence of an organic solvent to obtain an organic layer;
(b) separating and washing the organic layer;
(c) extracting the organic layer with an aqueous alkali;
(d) washing the alkali extract with the solvent used in step (a);
(e) separating the aqueous layer and agitating at room temperature to obtain the compound of formula (1).

3. A process as claimed in claim 2 wherein in step (b), the organic layer is washed with saturated bicarbonate solution of sodium or potassium.

4. A process as claimed in claim 2 wherein the oxidizing agent is a peracid selected from the group consisting of m-chloroperbenzoic acid, perbenzoic acid and peracetic acid.

5. A process as claimed in claim 2 wherein the concentration of oxidizing agent is in the range of 1 to 1.3 mole per mole of sulfide of formula (2).

6. A process as claimed in claim 2 wherein the solvent used is a halogenated hydrocarbon selected form the group consisting of chloroform and terachloroethane; or an alcohol selected from the group consisting of methanol, ethanol, propanol and butanol; or a mixture of any two or more thereof.

7. A process as claimed in claim 2 wherein the aqueous alkali is selected from the group consisting of hydroxides of Na, K and Li.

8. A process as claimed in claim 2 wherein the compound of formula (1) is a Ca and Mg salt and is obtained by treating Na, K or Li salt of tenatoprazole with halides of Mg and Ca or Mg(OR)₂Ca(OR)₂ wherein R is an alkyl group containing 1-4 carbon unit.
